(19) United States
(12) Patent Application Publication
(10) Pub. No.: US 2007/0255197 A1
(43) Pub. Date: Nov. 1, 2007

(54) Method of Treatment for Undesired Effect Following Transdermal or Topical Drug Delivery

(75) Inventors: Andrew Jonathan Humberstone, Victoria (AU); Nina Frances Wilkins, Victoria (AU); Igor Gonda, Victoria (AU)

Correspondence Address: FOLEY AND LARDNER LLP;SUITE 500, 3000 K STREET NW, WASHINGTON, DC, 20007, US

(73) Assignee: Acrux DDS Pty Ltd

(21) Appl. No.: 10/581,991

(22) PCT Filed: Dec. 9, 2004
(86) PCT No.: PCT/AU04/01738

§ 371 Date: May 8, 2007

(30) Foreign Application Priority Data
Dec. 10, 2003 (AU) 2003906842

(51) Int. Cl. A61M 37/00

(52) U.S. Cl. 604/30

(57) ABSTRACT

This invention relates to a method for inhibiting the percutaneous absorption of a physiologically active agent topically applied to a transdermal administration site of a subject, the method including the step of applying to skin of the subject at the transdermal administration site, a device (1, 7) comprising a membrane (8) for contacting the skin of the subject coated on the skin contacting side thereof (11) with a layer of an adhesive (10).

### FIELD

The present invention relates to a composition and method for reducing the effective transdermal dose of a topically applied drug for treatment or prophylaxis of undesired effects. The method the invention provides a method for inhibiting the release of a drug in to the systemic circulation in circumstances such as an overdose or where an adverse reaction is desired or expected after application.

### BACKGROUND

All drugs have the potential to be misused, whether legally prescribed by a doctor, purchased over-the-counter at the local drug store, or purchased illegally. Taken in combination with other drugs or with alcohol, even drugs normally considered safe can cause death or serious long term consequences. Accidental drug overdose may be the result of misuse of prescription medicines or commonly used medications like pain relievers and cold remedies. Symptoms differ depending on the drug taken.

While many victims of undesired drug effects recover without long term effects, there can be serious consequences. Some drug overdoses may cause the failure of major organs like the kidneys or liver, or failure of whole systems like the respiratory or circulatory systems.

Drugs have effects on the entire body. Generally, in an overdose, the effects of the drug may be a heightened level of the therapeutic effects seen with regular use. In overdose, side effects become more pronounced, and other effects can take place, which would not occur otherwise. Large overdoses of some medications cause only minimal effects, while smaller overdoses of other medications can cause severe effects, possibly death. Some overdoses may make worse a person's chronic disease. For example, an asthma attack or chest pains may be triggered.

Conventional means for administering antidotal agents to a human or animal suffering an undesired effect are to pump the stomach, thus mechanically remove unabsorbed drugs from the stomach, or to administer substances such as activated charcoal, to help bind drugs and reduce the amount absorbed into the blood. However, these methods are only applicable to treatment of oral overdose.

Administration of therapeutic agents through the skin (‘transdermal drug delivery’) has received increased attention because it is considered to not only provide a relatively simple dosage regime but also provide a relatively slow and controlled route for release of an agent into the systemic circulation. Thus, any adverse reaction or application error was traditionally remedied by removal of the transdermal system. However, with the current transdermal technology, the rate of transdermal uptake has dramatically increased, and risk of application error amplified along with it. Treatment of non-oral overdose currently requires administration of other medicines to reverse the effects or to prevent more harm.

Removal of drugs from the skin and/or systemic circulation offers several inherent clinical and patient advantages over the traditional remedies in that it is non-invasive, avoids further metabolism thus reducing the impact on the kidneys or liver, and can be self administered.

Structurally, the skin consists of two principle parts, a relatively thin outermost layer (the ‘epidermis’) and a thicker inner region (the ‘dermis’). The outermost layer of the epidermis (the ‘stratum corneum’) consists of flattened dead cells which are filled with keratin. The region between the flattened dead cells of the stratum corneum is filled with lipids which form lamellar phases that are responsible for the natural barrier properties of the skin. Epidermal thickness is remarkably constant over the body, except on the soles of the feet and the palms of the hand (Rushmer, et al., 1966, The Skin. Science 154 (3747), 343-348).

For effective transdermal delivery of a therapeutic agent that is applied to the surface of the skin (‘topical application’), the agent must be partitioned firstly from the vehicle into the stratum corneum, it must typically then be diffused within the stratum corneum before being partitioned from the stratum corneum to the viable epidermis and dermis and then into the bloodstream. Many transdermal systems now available rely on the rapid uptake into the stratum corneum and subsequent partitioning, thereby creating a reservoir of drug within the skin. Whilst the patch or transdermal system may be removed, the drug reservoir remains and will continue to partition into the systemic circulation.

There is a need for simple, effective removal of drugs from the skin and/or systemic circulation to reduce or avoid an undesirable effect.

### SUMMARY

The present invention arises from the inventor's studies of transdermal and topical formulations which contain penetration enhancers that enhance the percutaneous absorption of a physiologically active agent. The inventor's studies have shown that the release of physiologically active agent may be inhibited to ameliorate an undesired effect such as caused by overdose or adverse reaction.

The present invention provides a method for inhibiting the percutaneous absorption of a physiologically active agent topically applied to a transdermal administration site, the method including the step of applying to skin at the transdermal administration site a device comprising a membrane, with a coating of adhesive, applied to the skin contacting side thereof. The invention generally results in the treated subject receiving a serum concentration of topically applied drug which is less than would otherwise be provided. The membrane is preferably an occlusive or semi-permeable membrane.

According to the method of the invention the membrane device may be applied to the transdermal administration site to achieve a reduction in the drug serum profile of the animal to reduce or prevent the occurrence of undesired effects of the transdermal drug delivery. The membrane device is preferably applied to the whole of the transdermal drug application site and more preferably will include the surrounding area. In this way it is possible to inhibit the increase in the blood serum profile. Indeed in some cases we believe that the method of the invention leads to the drug being extracted from the skin to significantly reduce both the increase in blood level and the total dose of drug which would otherwise be administered. Moreover in many cases these effects of reduced blood level and total dosage occur even when the membrane device is applied after a significant period has elapsed since the topical application of the drug.

The present invention also provides a method for removal of physiologically active agent from the drug reservoir within the skin, the method including the step of applying a membrane to the transdermal drug administration site to reduce or eliminate the occurrence of undesired effect, wherein application of the membrane to the transdermal drug application site is used to extract the physiologically active agent from the drug reservoir within the skin.

The invention further provides the use of an adhesive in preparation of a membrane composite for treatment or prophylaxis of the effects of transdermal administration of a drug.

Conveniently, the membrane is coated with a layer of an adhesive on the skin contacting side of the membrane, which holds the membrane in place and prevents surface wrinkling. Preferably the membrane and adhesive are pliable and move with the body. Preferably the membrane is transparent and the free side of the membrane (that is the side remote from the side applied to the body) is resistant to liquids, thus enabling the individual to shower or bathe.

### DETAILED DESCRIPTION

Before describing the present invention in detail, it is to be understood that this invention is not limited to specific drug delivery systems, device structures, enhancers or carriers, as such may vary. It is also to be understood that the terminology used is for the purpose of describing particular embodiments only, and is not intended to be limiting.

In describing the present invention, the following terminology will be used in accordance with the definitions set out below.

The terms “topical” and “transdermal” are used herein in the broadest sense to refer to administration of a drug to the skin surface or mucosal membrane of an animal, including humans, so that the drug passes through the skin tissue and/or into the animal's blood stream, thereby providing a local or systemic effect. The term transdermal is intended to include transmucosal drug administration i.e. administration of a drug to the mucosal surface of an animal so that the drug passes through the mucosal tissue and into the blood stream. Unless otherwise stated or implied, the terms topical drug delivery and transdermal drug delivery are used interchangeably.

The term “skin-reservoir” is used herein in its broadest sense to refer to a depot or deposit of active agent and dermal penetration enhancer within the epidermis, whether it is intra-cellular (within keratinocytes) or inter-cellular.

The term “stratum corneum” is used herein in its broadest sense to refer to the outer layer of the skin, which is comprised of (approximately 15) layers of terminally differentiated keratinocytes made primarily of the proteinaceous material keratin arranged in a ‘brick and mortar’ fashion with the mortar being comprised of a lipid matrix made primarily from cholesterol, ceramides and long chain fatty acids. The stratum corneum creates the rate-limiting barrier for diffusion of the active agent across the skin.

As mentioned previously, the present invention provides a method for reducing or eliminating the percutaneous absorption of a physiologically active agent thereby preventing elevated drug serum concentrations within the bloodstream of an animal suffering from an undesired effect of a transdermally administered drug, the method including the step of applying an occlusive or semi-permeable membrane, coated with a layer of an adhesive, to the transdermal application site.

Typically, the membrane will comprise of a suitable elastic, occlusive or semi-permeable layer such as films of polyurethane and ethyl vinyl acetate copolymers, hydrocolloid or cellulose.

Conveniently, the membrane of the current invention comprises an adhesive layer on one side. The adhesive layer will be comprised of a material that is permeable to the drug intended to be extracted. Examples of suitable materials for the adhesive layer include acrylics, polyethylenes, polysiloxanes, polyisobutylenes, polyacrylates, polyurethanes, plasticized ethylene vinyl acetate copolymers, tacky rubbers such as polyisobutene and the like.

The membrane will typically be less than 5 mm thick, more preferably less than 2 mm thick.

A benefit of the method of the present invention is that, in the event of an undesired effect, the systemic or local uptake may be prevented or reduced, thereby reducing or eliminating the effect and potential side effects. In contrast with other overdose treatments, the method of the present invention can be self administered safely, quickly and efficiently without encountering the gastro irritation problems of traditional remedies.

The dose of transdermal administration is preferably reduced by at least 10% more preferably by at least 20% and most preferably by at least 30%. The reduction in dose can be determined by measuring the proportion of the dose extracted by the membrane.

The method of the present invention may be applied to inhibit or reduce any transdermal or topical delivery system comprising a physiologically active agent which can be delivered through the skin with or without the assistance of a dermal penetration enhancer. A list of suitable physiologically active agents includes, but is not limited to:

Antidiarrhoeals such as diphenoxylate, loperamide and hyoscyamine.

Antihypertensives such as hydralazine, minoxidil, captopril, enalapril, clonidine, prazosin, debrisoquine, diazoxide, guanethidine, methyldopa, reserpine, trimetaphan and lacidipine.

Calcium channel blockers such as diltiazem, felodopine, amlodipine, nitrendipine, nifedipine and verapamil.

Antiarrhythmics such as amiodarone, flecainide, disopyramide, procainamide, mexiletene and quinidine.

Anti-angina agents such as glyceryl trinitrate, erythritol tetranitrate, pentaerythritol tetranitrate, mannitol hexanitrate, perhexilene, isosorbide dinitrate and nicorandil.

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

Cardiotonic glycosides such as digoxin and other cardiac glycosides and theophylline derivatives.

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine.

Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol.

Anti-migraine preparations such as ergotamine, dihydroergotamine, methysergide, pizotifen and sumatriptan.

Anticoagulants and thrombolytic agents such as warfarin, dicoumarol, low molecular weight heparins such as enoxaparin; streptokinase and its active derivatives. Haemostatic agents such as aprotinin, tranexamic acid and protamine.

Analgesics, antipyretics including the opioid analgesics such as buprenorphine, dextromoramide, dextropropoxyphene, butorphanol, fentanyl, ketamine, alfentanil, sufentanil, hydromorphone, methadone, morphine, oxycodone, papaveretum, pentazocine, pethidine, phenoperidine, codeine and dihydrocodeine. Others include acetylsalicylic acid (aspirin), paracetamol, rizatriptin, sumatriptan, zolmitriptan and phenazone.

Hypnotics and sedatives such as the barbiturates, amylobarbitone, butobarbitone and pentobarbitone and other hypnotics and sedatives such as ketamine, choral hydrate, chlormethiazole, hydroxyzine and meprobamate.

Anti-anxiety agents such as the benzodiazepines, alprazolam, bromazepam, chlordiazepoxide, clobazam, chlorazepate, diazepam, flunitrazepam, flurazepam, lorazepam, nitrazepam, buspirone, oxazepam, temazepam and triazolam.

Neuroleptic and antipsychotic drugs such as the phenothiazines, chloropromazine, fluphenazine, pericyazine, perphenazine, promazine, thiopropazate, thioridazine and trifluoperazine and the butyrophenones, droperidol and haloperidol and the other antipsychotic drugs such as pimozide, thiothixene, olanzapine and lithium.

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin reuptake inhibitors such as fluoxetine, paroxetine, citalopram, fluvoxamine and sertraline.

CNS stimulants such as caffeine and methyl phenidate.

Anti-Alzheimer's agents such as tacrine, zinc chelators such as phenanthrolines and their derivatives, such as 1,10 phenanthroline, aryl proprionic acids and their derivatives, such as ibuprofen and flurbiprofen.

Anti-Parkinson agents such as apomorphine, amantadine, benserazide, carbidopa, rivastigmine, levodopa, benztropine, biperiden, benzhexol, procyclidine, pergolide, ropinirole and dopamine-2 agonists such as S(−)-2-(N-propyl-N-2-thienylethylamino)-5-hydroxytetralin (N-0923).

Anticonvulsants such as phenyloin, valproic acid, primidone, phenobarbitone, methylphenobarbitone and carbamazepine, ethosuximide, methsuximide, phensuximide, sulthiame and clonazepam.

Antiemetics, antinauseants such as the phenothiazines, prochloperazine, thiethylperazine and 5HT-3 receptor antagonists such as ondansetron, tropisetron and granisetron and others such as dimenhydrinate, diphenhydramine, metoclopramide, domperidone, hyoscine, hyoscine hydrobromide, hyoscine hydrochloride, clebopride and brompride.

Non-steroidal anti-inflammatory agents including their racemic mixtures or individual enantiomers where applicable, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol and ketoralac, salicylamide, flufenisal, salsalate, triethanolamine salicylate, aminopyrine, antipyrine, oxyphenbutazone, apazone, cintazone, flufenamic acid, clonixeril, clonixin, meclofenamic acid, flunixin, coichicine, demecolcine, allopurinol, oxypurinol, benzydamine hydrochloride, dimefadane, indoxole, intrazole, mimbane hydrochloride, paranyline hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Anti-rheumatoid agents such as penicillamine, aurothioglucose, sodium aurothiomalate, methotrexate and auranofin.

Muscle relaxants such as baclofen, diazepam, cyclobenzaprine hydrochloride, dantrolene, methocarbamol, orphenadrine and quinine.

Agents used in gout and hyperuricaemia such as allopurinol, coichicine, probenecid and sulphinpyrazone.

Oestrogens such as oestradiol, oestriol, oestrone, ethinyloestradiol, mestranol, stilboestrol, dienoestrol, epioestriol, estropipate and zeranol.

Progesterone and other progestagens such as allyloestrenol, dydrgesterone, lynoestrenol, norgestrel, norethyndrel, norethisterone, norethisterone acetate, gestodene, levonorgestrel, medroxyprogesterone and megestrol.

Antiandrogens such as cyproterone acetate, flutamide and danazol.

Anti-oestrogens such as tamoxifen and epitiostanol and the aromatase inhibitors, exemestane and 4-hydroxy-androstenedione and its derivatives.

Androgens and anabolic agents such as calusterone, clostebol acetate, dehydroepiandrostenedione (DHEA), dihydrotestosterone (DHT), dromostanolone propionate, drostanolone, enanthate, ethylestrenol, fluoxymesterone, furazabol, methandriol, methandrostenolone, methyltestosterone, nandrolone decanoate, nandrolone oxandrolone, oxymetholone, nandrolone phenpropionate, stanozolol, testolactone, testosterone, testosterone cypionate, testosterone propionate, testosterone trenbolone acetate, 7-methyl-19-testosterone (MENT), and 17-α-methyl-19-nortestosterone.

Anti-alopecia agents such as minoxidil, cromakalin, pinacidil, naminidil, diphenylcyclopropenone, tricomin, and those compounds selected from the classes of s-triazines, benzopyrans, pyridinopyrans and thiane-1-oxides.

5-alpha reductase inhibitors such as finasteride, turosteride, LY-191704, MK-386 and dutasteride.

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide.

Further examples of steroidal anti-inflammatory agents include cortodoxone, fluoracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

Pituitary hormones and their active derivatives or analogues such as corticotrophin, thyrotropin, follicle stimulating hormone (FSH), luteinising hormone (LH) and gonadotrophin releasing hormone (GnRH).

Hypoglycaemic agents such as insulin, chlorpropamide, glibenclamide, gliclazide, glipizide, tolazamide, tolbutamide and metformin.

Thyroid hormones such as calcitonin, thyroxine and liothyronine and antithyroid agents such as carbimazole and propylthiouracil.

Other miscellaneous hormone agents such as octreotide.

Pituitary inhibitors such as bromocriptine.

Ovulation inducers such as clomiphene.

Anti-muscarinic agents including atropine, belladonna alkaloids, benzatropine (benztropine), biperiden, cyclopentolate, dicycloverine (dicyclomine), flavoxate, homatropine, hyoscine, ipratropium, orphenadrine, oxitropium, oxybutynin, procyclidine, propantheline, propiverine, tiotropium, tolterodine, trihexyphenidyl (benzhexol), tropicamide and trospium.

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetamide, ethacrynic acid and frusemide and potassium sparing diuretics, spironolactone, amiloride and triamterene.

Antidiuretics such as desmopressin, lypressin and vasopressin including their active derivatives or analogues.

Obstetric drugs including agents acting on the uterus such as ergometrine, oxytocin and gemeprost.

Prostaglandins such as alprostadil (PGE1), prostacyclin (PGI2), dinoprost (prostaglandin F2-alpha) and misoprostol.

Antimicrobials including the cephalosporins such as cephalexin, cefoxytin and cephalothin.

Penicillins such as amoxicillin, amoxicillin with clavulanic acid, ampicillin, bacampicillin, benzathine penicillin, benzylpenicillin, carbenicillin, cloxacillin, methicillin, phenethicillin, phenoxymethylpenicillin, flucloxacillin, mezlocillin, piperacillin, ticarcillin and aziocillin.

Tetracyclines such as minocycline, chlortetracycline, tetracycline, demeclocycline, doxycycline, methacycline and oxytetracycline and other tetracycline type antibiotics.

Aminoglycosides such as amikacin, gentamicin, kanamycin, neomycin, netilmicin and tobramycin.

Antifungals such as butenafine, butoconazole, clioquinol, itraconazole, lanoconazole, neticonazole, tioconazole, terconazole, ciclopirox olamine, amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione.

Quinolones such as nalidixic acid, cinoxacin, ciprofloxacin, enoxacin and norfloxacin.

Sulphonamides such as phthalylsulphthiazole, sulfadoxine, sulphadiazine, sulphamethizole and sulphamethoxazole.

Sulphones such as dapsone.

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, tinidazole, fusidic acid and trimethoprim; 2-thiopyridine N-oxide; halogen compounds, particularly iodine and iodine compounds such as iodine PVP complex and diiodohydroxyquin; hexachlorophene; chlorhexidine; chloroamine compounds; benzoylperoxide.

Anti-tuberculosis drugs such as ethambutol, isoniazid, pyrazinamide, rifampicin and clofazimine.

Antimalarials such as primaquine, pyrimethamine, chloroquine, hydroxychloroquine, quinine, mefloquine and halofantrine.

Antiviral agents such as acyclovir and acyclovir prodrugs, famciclovir, zidovudine, didanosine, stavudine, lamivudine, zalcitabine, saquinavir, indinavir, ritonavir, n-docosanol, tromantadine and idoxuridine.

Anthelmintics such as mebendazole, thiabendazole, niclosamide, praziquantel, pyrantel embonate and diethylcarbamazine.

Cytotoxic agents such as plicamycin, cyclophosphamide, dacarbazine, fluorouracil and its prodrugs, methotrexate, procarbazine, 6-mercaptopurine and mucophenolic acid.

Anorectic and weight reducing agents including dexfentturamine, fenfluramine, diethylpropion, mazindol and phentermine.

Agents used in hypercalcaemia such as calcitriol, dihydrotachysterol and their active derivatives or analogues.

Antitussives such as ethylmorphine, dextromethorphan and pholcodine.

Expectorants such as acetylcysteine, bromhexine, emetine, guaiphenesin, ipecacuanha and saponins.

Decongestants such as phenylephrine, phenylpropanolamine and pseudoephedrine.

Bronchospasm relaxants such as ephedrine, fenoterol, orciprenaline, rimiterol, salbutamol, sodium cromoglycate, cromoglycic acid and its prodrugs, terbutaline, ipratropium bromide, salmeterol and theophylline and theophylline derivatives.

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

Local anaesthetics such as bupivacaine, amethocaine, lignocaine, cinchocaine, dibucaine, mepivacaine, prilocalne and etidocaine.

Neuromuscular blocking agents such as suxamethonium, alcuronium, pancuronium, atracurium, gallamine, tubocurarine and vecuronium.

Smoking cessation agents such as nicotine, bupropion and ibogaine.

Insecticides and other pesticides which are suitable for local or systemic application.

Dermatological agents, such as vitamins A and E, vitamin E acetate and vitamin E sorbate.

Nutritional agents, such as vitamins, essential amino acids and essential fats.

Keratolytics such as the alpha-hydroxy acids, glycolic acid and salicylic acid.

Psychic-energisers, such as 3-(2-aminopropyl)indole, 3-(2-aminobutyl)indole, and the like.

Anti-acne agents such as containing isotretinoin, tretinoin and benzoyl peroxide.

Anti-psoriasis agents such as containing etretinate, cyclosporin and calcipotriol.

Anti-itch agents such as capsaicin and its derivatives such as nonivamide [Tsai, et al. Drug. Dev. Ind. Pharm., 20 (4), 719, 1994].

Anticholinergic agents, which are effective for the inhibition of axillary sweating and for the control of prickly heat. The antiperspirant activity of agents such as methatropine nitrate, propantheline bromide, scopolamine, methscopolamine bromide, and the new class of soft antiperspirants, quaternary acyloxymethyl ammonium salts.

Other physiologically active peptides and proteins, small to medium sized peptides, e.g., vasopressin and human growth hormone.

The membrane may comprise one or more solvent reservoirs. The portion of membrane between the reservoir and the adhesive layer may be a one-way membrane. The one way membrane may function so that drug extracted from the systemic circulation and/or skin layers permeates through the one-way membrane and into the solvent reservoir.

The membrane is preferably administered to the application site within 4 days of transdermal drug application, more preferably within 24 hours of transdermal drug application, and most preferably within 1 hour of transdermal drug application.

Preferred aspects of the invention are described in more detail with reference to the attached drawings.

In the accompanying drawings:

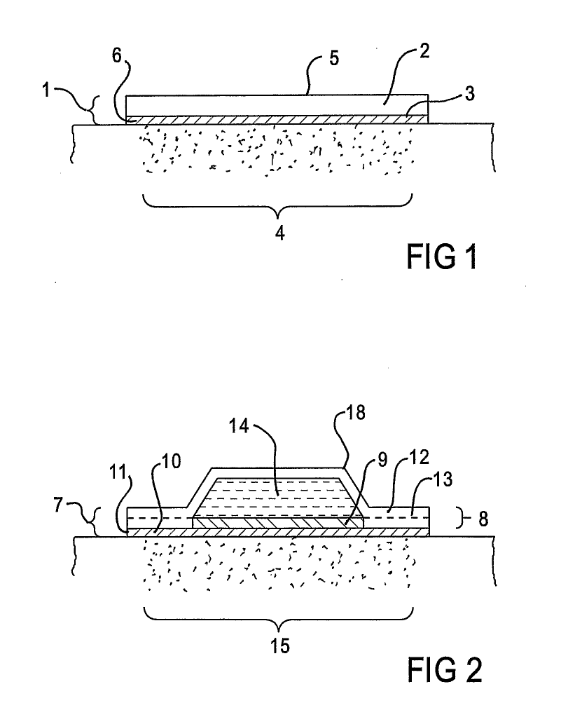

FIG. 1 is a schematic drawing showing a cross section of an occlusive membrane which has been applied to a topically treated area of skin in accordance with a preferred embodiment of the present invention.

FIG. 2 is a schematic drawing showing a cross section of an occlusive membrane applied to a topically treated area of skin in accordance with an alternative, though less preferred, embodiment of the invention.

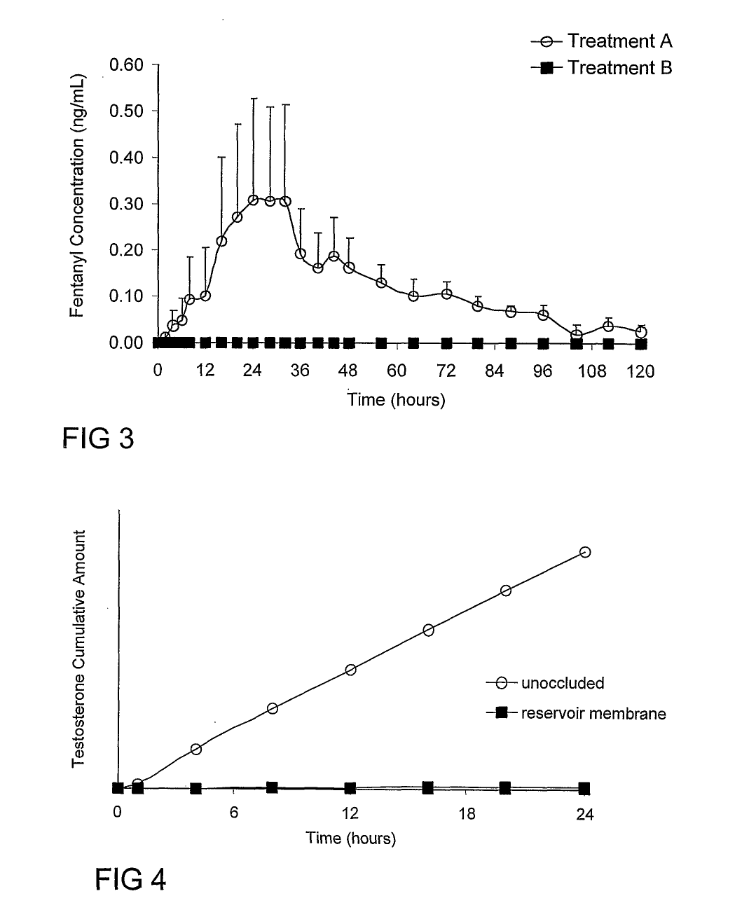

FIG. 3 is a graph showing the mean (±SEM; n=5) serum concentration-time profiles after application of treatment A (a single dose of 140 μl fentanyl formulation) or treatment B (a single dose of 140 μl fentanyl formulation followed by occlusion for 24 hours). Values below the assay LOQ (0.1 ng/ml) have been omitted.

FIG. 4 is a graph showing the cumulative amount of testosterone diffused across skin with and without solvent reservoir membrane application.

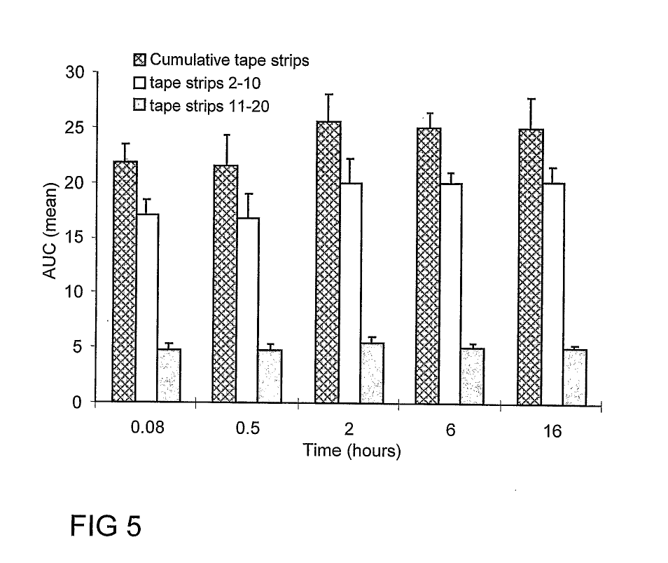

FIG. 5 is a bar chart showing the fentanyl distribution between the upper and lower stratum corneum at different times up to 16 hours after spray application of a fentanyl composition containing demal penetration enhancer.

Referring to the drawings FIG. 1 shows a membrane assembly (1) in accordance with a preferred embodiment of the invention comprising an occlusive membrane (2) having a skin side surface (3) to be applied adjacent an area of topically treated skin (4) and a free surface (5) remote there from. The skin side surface (3) is provided with an adhesive layer (6) formed of a suitable skin safe adhesive.

In the method of the invention the membrane assembly (1) is used in circumstances where it is necessary to terminate or reduce transdermal administration following topical application of the active in a transdermal formulation. The need to terminate or reduce transdermal administration may arise as a result of learning after the event that an overdose has been topically applied or the occurrence of adverse reaction or other any other factor which may lead to it being considered desirable to reduce the dose which would otherwise result from the initial topical application. The area of topical application (4) is identified and the membrane assembly applied preferably with sufficient dimensions to at least substantially cover the area of topical application (4). The membrane assembly (1) is applied to the skin in the area of application (4) so that the adhesive layer (6) on the skin contacting side (3) of the membrane (3) makes adhering contact with the skin (4). Pressure, such as firm hand pressure, is preferably applied to the free surface (5) of the occlusive membrane (2) to urge the membrane assembly (1) into a uniformly adhering contact with the skin (4).

Referring to FIG. 2 the alternative membrane assembly (7) comprises a multilayer semi permeable membrane (8) comprising a skin side layer (9) which is selectively permeable to allow the ingress of the topically applied composition. The skin side layer (9) is provided with a skin contacting adhesive (10) on the skin side thereof (11) and is bonded to a second layer (12), which may be impermeable, on the remote side (13). One or more reservoirs of solvent (14) are provided between the first (9) and second layers (12). The solvent reservoir (14) may provide an osmotic potential promoting absorption of topical composition through the first (selectively permeable) layer (9). Preferably the first membrane layer (9) is substantially impermeable to the egress of solvent from the solvent reservoir (14). In this embodiment the method of the invention includes applying the membrane assembly to the area of skin to which the topical application has occurred (15). The assembly (7) is applied so that the adhesive layer (10) is brought into adhering contact with the skin (15) and the adhesion is preferably made substantially uniform by application of pressure (e.g. hand pressure) to the outer side (18) of the membrane assembly.

In accordance with a preferred embodiment we provide a method of reducing the effect of overdose via transdermal administration of a physiologically active agent to a site of skin of a subject to form a reservoir of physiologically active agent in the skin the method comprising providing a membrane assembly for contacting the site of skin the membrane assembly comprising (a) selectively permeable membrane for making contact with the skin to allow ingress of physiologically active agent and provided with an adhesive layer on the skin side thereof, (b) a backing layer and (c) a reservoir of solvent between the backing layer and membrane wherein the physiologically active agent is at least partly soluble in the solvent and preferably (d) a solvent impermeable layer adjacent the side of said membrane remote from the adhesive; and applying the adhesive layer of the membrane assembly to the site of transdermal administration wherein the physiologically active agent is extracted from the skin into the membrane assembly.

The choice of solvent used in the reservoir may be selected on the basis of the particular drug previously administered in order to achieve the desired extraction effect, since the drug must be at least partially soluble in the selected solvent. Preferably, the solvent is an alcohol, alkane, ether, ketone, chlorinated hydrocarbon or nitrile. More preferably the solvent is aliphatic C₁-C₄. Most preferably the solvent is selected from the group consisting of ethanol and its derivatives, methanol, chloroform, isopropyl alcohol or a mixture of two or more of the aforementioned solvents.

The amount of solvent used will depend on the period which has elapsed following initial topical application and the physico chemical properties of the solvent and drug and the area of application of the drug.

The occlusive or semi-permeable membrane is applied to the transdermal application site preferably within 4 days, more preferably within 24 hours and most preferably within 1 hour.

The occlusive or semi-permeable membrane is applied to as much of the transdermal drug application site as possible, including the surrounding area and remains in place preferably for 12 hours, more preferably for 24 hours.

Conveniently, the occlusive or semi-permeable membrane is coated with a layer of an adhesive, to achieve a fixed and secure positioning on the skin.

In a further embodiment the invention provides for a method of solvent extraction whereby the drug application site may be swabbed with a solvent mix and/or the occlusive or semi-permeable membrane contains a reservoir solvent. In a particularly preferred form of the invention the solvent is a lower alcohol, more preferably methanol or chloroform, or a mixture thereof.

The amount of drug prevented from absorption and/or extracted will depend on a number of factors and will vary from subject to subject and depend on the particular drug administered and the length of time prior to application of the occlusive or semi-permeable membrane. The desired effect of the present invention is such that the quantity of drug present will not exceed a rate of release that reaches levels that provide the undesired effect.

The method of the invention may be used to reduce the level of administration of active agents from a range of transdermal formulations administered via patches, sprays or other topical methods. Such formulates will generally comprise an active agent, a carrier which preferably includes a volatile solvent and optionally a penetration enhancer. The compositions will preferably contain a penetration enhancer. Examples of such compositions are disclosed in U.S. Pat. No. 6,229,900 the contents of which are herein incorporated by reference. Examples of suitable carriers and penetration enhancers are described in U.S. Pat. No. 6,229,900.

Examples of dermal penetration enhancers include fatty acids, fatty acid esters, fatty alcohols, glycols and glycol esters, 1,3-dioxolanes and 1,3-dioxanes, macrocyclic ketones containing at least 12 carbon atoms, oxazolidinones and oxazolidinone derivatives, alkyl-2-(N,N-disubstituted amino)-alkanoate esters, (N,N-disubstituted amino)-alkanol alkanoates, sunscreen esters and mixtures thereof. More preferably the dermal penetration enhancer is selected from the list including oleic acid, oleyl alcohol, cyclopentadecanone (CPE-218™), sorbitan monooleate, glycerol monooleate, propylene glycol monolaurate, polyethylene glycol monolaurate, 2-n-nonyl 1,3-dioxolane (SEPA™), dodecyl 2-(N,N-dimethylamino)-propionate (DDAIP) or its salt derivatives, 2-ethylhexyl 2-ethylhexanoate, isopropyl myristate, dimethyl isosorbide, 4-decyloxazolidinon-2-one (SR-38™, TCPI, Inc.), 3-methyl-4-decyloxazolidinon-2-one, octyl dimethyl-para-aminobenzoate, octyl para-methoxycinnamate, octyl salicylate and mixtures thereof.

Examples of volatile solvents include skin tolerant solvents such as ethanol isopropanol and aerosol propellants such as diethylether, hydrofluorocarbons and the like.

The transdermal formulation may have a rapid drying time for example up to 3 minutes. Notwithstanding that formulations with a rapid drying time are more effective in driving the composition into the epidermis we have found that the method of the invention allows a significant reduction in dose to be achieved with such compositions.

The invention will now be described with reference to the following examples. It is to be understood that the examples are provided by way of illustration of the invention and that they are in no way limiting to the scope of the invention.

### EXAMPLE 1

The objectives of this pharmacokinetic study were to determine the pharmacokinetics of fentanyl after single dosing in healthy volunteers and to compare the pharmacokinetic profile of the fentanyl profile with and without occlusion of the application site.

Method

The study was a parallel design. In the first stage 5 healthy volunteers received treatment A. The second stage 5 healthy volunteers received treatment B. The treatments consisted of a single application of 140 μl fentanyl formulation applied to the abdomen, with and without occlusion of the application site. Serum fentanyl concentration profiles were measured over 72 hours after administration of transdermal fentanyl. A validated GC/MS assay was used to analyse fentanyl concentrations in the serum samples.

Study Treatments

Treatment A: Fentanyl formulation (140 μl) containing 7.5% fentanyl and 5.0% Octisalate applied to the lateral abdomen.
Treatment B: Fentanyl formulation (140 μl) containing 7.5% fentanyl and 5.0% Octisalate applied to the lateral abdomen and then covered by an occlusive dressing to the whole area within 2 minutes (polyurethane membrane coated with a layer of an acrylic adhesive) for 24 hours.
   Result

Treatment B (fentanyl formulation with occlusion) produced very low levels of serum fentanyl (FIG. 3). Values below the assay LOQ (0.1 ng/ml) have been omitted from the graphs. The values were significantly lower than the levels seen after application of Treatment A (fentanyl formulation without occlusion). This indicates that occlusion of the application site with polyurethane membrane patches, coated with a layer of an acrylic adhesive, significantly reduces absorption from the skin reservoir.

TABLE 1
Mean (±S.E.M.) pharmacokinetic parameters
following application of treatments A and B.

| | AUC_{0-72 h} | | |
|---|---|---|---|
| Treatment | (ng/mL · h) | Cₘₐₓ (ng/mL) | tₘₐₓ (hours) |
| A (without occlusion) | 11.89 ± 7.00 | 0.33 ± 0.21 | 36 ± 9.12 |
| B (with occlusion) | 0.00 ± 0.00 | 0.00 ± 0.00 | 0 ± 0.00 |

### EXAMPLE 2

The occlusive or semi-permeable membrane may contain a reservoir solvent. The choice of solvent used in the reservoir may be selected on the basis of the particular drug previously administered in order to achieve the desired extraction effect. The aforementioned examples are not meant to be limiting and it is envisaged that combinations of solvents could also be used to obtain the desired pharmacological effect, for example on a weight basis

| Solvent | Ratio (% w/w) |
|---|---|
| Methanol:Chloroform | 20-80:20-80 |

FIG. 4 depicts the in-vitro diffusion profile that may be obtained by application of a solvent reservoir membrane immediately following undesirable administration of testosterone.

### EXAMPLE 3

This example determines the amount of fentanyl absorbed by an adhesive membrane during the procedure described in Example 1.

Materials and Methods

HPLC Conditions

The following HPLC conditions were used to measure the content of fentanyl in the membrane. The mobile phase constituted the same components as for the HPLC Fentanyl diffusion assay. The flow condition is 1 ml/min isocratic at 75% A, 25% B. The “Previal C18” brand column 250×4.6 mm, (available from Alltech) was used.

The condition was validated. The standard curve had concentrations between 0.1-2 μg/ml fentanyl.

Extraction Procedure

1. The patch was placed in glass vial;

2. A known amount (40 ml) of ethanol (100%) was added to the vial;

3. The vial was sonicated for 40 minutes;

4. The solution was filtered with Millipore Millex HN Nylon 0.45 μm filter; and

5. Filtrates were diluted 1 in 5 parts with MilliQ water prior to injection into HPLC. Further dilution may require if solution is too concentrated.

Results and Discussion

Two patches from the same subject were obtained and analysed. The amount of fentanyl obtained from the 2 patches was 5.85 mg.

In the trial, 140 μL of 7.5% fentanyl solution delivered to each subject, i.e. 10.2 mg fentanyl. The amount recovered from the patch was therefore 56.8% of the delivered dose.

### EXAMPLE 4

The objective of this study was to determine the distribution of fentanyl across human stratum corneum at various exposure times. The data for the example is taken from the PhD of B. T. Traversa 2004, Victorian College of Pharmacy.

Method

Full-thickness human skin samples were prepared by removing subcutaneous tissue from the underside of the dermal membrane using a stainless steel surgical blade. The stratum corneum surface was rinsed with Milli-Q™ water and gently wiped in order to remove any surface contamination.

The skin was laid flat, stratum corneum side up, and dissected into 1×5 cm (width×length) sections (n=5) using a stainless steel surgical blade. Each section was laid flat, dermal side up, and quick-dry super glue (Selleys® “Fix'n'Go Supa Glue”, Selleys, Australia) was blotted onto the ventral surface of the dermis. The skin sections were immediately mounted, stratum corneum side up, on to 1×7 cm pieces of cardboard (the excess 2 cm in length was to enable handling of the samples). Gentle pressure was then applied to the skin for approximately 10 sec to ensure contact with the cardboard.

The formulation was prepared by dissolving 5% w/v fentanyl, with 5% w/v octisalate, in 95% v/v ethanol and applied (5 μl/cm2) to the stratum corneum surface of each skin, sample. After a pre-determined exposure time (5 min (0.08 h), 0.5, 2, 6, or 16 h), excess formulation was removed from the stratum corneum surface by swabbing with cotton buds.

The cotton buds were placed in a glass vial and a 10 ml aliquot of 100% methanol was added. The contents were sealed with a teflon-lined lid, vortexed for 30 sec, and then placed on a horizontal roller mixer for gentle mixing over 16 h at ambient temperature (Stage 1). At the end of the 16 h extraction period, the sample was vortexed for 30 sec, the cotton buds were then transferred to an empty glass vial and 5 ml of methanol was added. The sample was vortexed for 30 sec, then placed on the horizontal roller mixer for 8 h at ambient temperature (Stage 2). At the end of the 8 h period, the methanol extract retained from Stage 1 was added to the sample. The sample was then vortexed for 1 min and 5 ml of the extract was centrifuged at 3500 rpm for 15 min at 25° C. A 1 ml aliquot of the supernatant was diluted to 10 ml with methanol for HPLC/UV analysis.

The stratum corneum was then progressively removed by sequential adhesive tape stripping. Sections of 1.2×5 cm pieces of polyester adhesive tape were applied to the stratum corneum surface and a constant pressure of 240 g/cm2 was applied to the tape for 5 sec.

After the tape was applied, it was removed from the stratum corneum surface. The tape stripping procedure was repeated 20 times in order to remove most of the stratum corneum. The tape strips were placed adhesive side down onto filter paper and the samples were then cut to size and individually placed into 15 ml glass centrifuge vials. Aliquots of HPLC grade methanol 100% were added to the tape strip samples. 10 ml was added to each of the first 10 tape strips, and 5 ml was added tape strips 11 to 20. The samples were vortexed for 30 seconds and then placed in a 25° C. shaking water bath, where they were continuously shaken at 15 strokes/min for 24 h.

At the end of the 24 h period, the samples were removed from the water bath and vortexed for 30 sec. The tape strips and filter paper were removed from the vials and discarded. The extracts were then centrifuged at 3500 rpm for 15 min at 25° C. After centrifugation, a 1 ml aliquot was taken from each of the extracts of tape strips 1 and 2 and diluted to 10 ml with methanol for HPLC/UV analysis. The extracts from tape strips 3 to 20 were analysed undiluted.

Results

In general, fentanyl concentrations declined exponentially across the stratum corneum with exposure times between 0.08 to 6 h. After 16 h of exposure, concentrations became more linearly distributed across the stratum corneum. When fentanyl was applied with octyl salicylate, concentrations within the upper layers of the stratum corneum remained relatively constant over the various exposure times.

Referring to FIG. 5. Fentanyl distribution profiles following the application of fentanyl with octyl salicylate. AUC's were calculated for the entire stratum corneum (i.e. stratum corneum removed by tape strips 2-20, AUCx/L2→20) the upper stratum corneum (i.e. stratum corneum removed by tape strips 2-10, AUCx/L2→10) and the lower stratum corneum (i.e. stratum corneum removed by tape strips 11-20, AUCx/L11→20).

As shown in FIG. 5 a significant amount of fentanyl remains at the skin surface and within the upper stratum corneum layers after extended exposure times. The method of the present invention will still exert a significant effect on fentanyl partitioning after longer exposure times.

1. A method for inhibiting the percutaneous absorption of a physiologically active agent topically applied to a transdermal administration site of a subject, the method including the step of applying to skin of the subject at the transdermal administration site, a device comprising a membrane for contacting the skin of the subject coated on the skin contacting side thereof with a layer of an adhesive.

2. A method according to claim 1 wherein the membrane device is applied to the transdermal administration site of a subject who has received at least one of (a) an overdose of transdermally applied physiologically active agent and (b) adverse side effects from the physiologically active agent, wherein the dose of physiologically active agent transferred to the blood stream is thereby reduced.

3. A method according to claim 1 wherein the membrane is applied to the whole of the transdermal application site.

4. A method according to claim 1 wherein the physiologically active agent is administered so as to form a reservoir of the physiologically active agent in the skin and the application of said membrane device results in the physiologically active agent being extracted from the skin to significantly reduce the total dose of drug which would otherwise be administered transdermally.

5. A method for removal of physiologically active agent from the reservoir thereof within the skin of a subject following transdermal administration of the physiologically active agent to a site on the skin of the subject the method including the step of applying a device comprising a membrane to the site of transdermal administration of the pharmaceutically active agent.

6. A method according to claim 5 wherein the membrane is coated with a layer of an adhesive on the skin contacting side of the membrane for adhering the membrane to the site of transdermal administration.

7. A method according to claim 1 wherein the membrane device comprises of an elastic, occlusive or semi-permeable layer selected from polyurethane polymers, ethylene vinyl acetate copolymers, hydrocolloid and cellulosic membranes.

8. A method according to claim 1 wherein the adhesive layer is permeable to the physiologically active agent and is selected from the group consisting of acrylics, polyethylenes, polysiloxanes, polyisobutylenes, polyacrylates, polyurethanes, plasticized ethylene vinyl acetate copolymers and tacky rubbers.

9. A method according to claim 1 wherein the membrane is less than 2 mm thick.

10. A method according to claim 1 wherein the membrane is applied to the site of transdermal administration within 24 hours of transdermal application of the physiologically active agent.

11. A method according to claim 1 wherein an overdose of physiologically active agent has been topically applied to the site of skin prior to the membrane being applied thereto.

12. A method according to claim 1 wherein the membrane device is an assembly further comprising at least one layer on the side of said membrane remote from the side applied to the skin and wherein a reservoir of solvent is provided between said at least one layer and said membrane wherein said active agent is at least partially soluble in the solvent.

13. A method according to claim 12 wherein the solvent is selected from the group consisting of alcohols, alkanes, ethers, ketones, chlorinated hydrocarbons and nitrites.

14. A method according to claim 12 wherein the solvent is selected from the group consisting of ethanol and its derivatives, methanol, chloroform, isopropyl alcohol and mixture of two or more thereof.

15. A method according to claim 12 wherein the membrane remains adhered to the skin at the site of transdermal administration for a period of at least 12 hours.

16. A method of reducing the effect of overdose via transdermal administration of a physiologically active agent to a site of skin of a subject to form a reservoir of physiologically active agent in the skin the method comprising providing a membrane assembly for contacting the site of skin the membrane assembly comprising (a) selectively permeable membrane for making contact with the skin to allow ingress of physiologically active agent and provided with an adhesive layer on the skin side thereof, (b) a backing layer and (c) a reservoir of solvent between the backing layer and membrane wherein the physiologically active agent is at least partly soluble in the solvent and preferably (d) a solvent impermeable layer adjacent the side of said membrane remote from the adhesive; and applying the adhesive layer of the membrane assembly to the site of transdermal administration wherein the physiologically active agent is extracted from the skin into the membrane assembly.

17. A method according to claim 1 wherein the physiologically active agent comprises at least one selected from the group consisting of anti-diarrhoeals, anti-hypertensives, calcium channel blockers, anti-arrhythmics, anti-angina agents, beta-adrenergic blocking agents, cardiotonic glycosides, adrenergic stimulants, vasodilators, anti-migraine preparations, anticoagulants, thrombolytic agents, analgesics, hypnotics, anti-anxiety agents, neuroliptic agents anti-psychoticagents, antidepressants, CNS stimulates, anti-Alzheimer agents, anti-Parkinson agents, anticonvulsants, anti-emetics, non-steroidal anti-inflammatory agents, anti-rheumatoid agents, muscle relaxant agents for treatment of gout, agents for treatment of hyperuricaemia, oestrogens, progesterone, anti-androgens, anti-oestrogens, androgens, anti-alopecia agents, 5-alpha reductase inhibitors, carbosteroids, pituitary hormones, hypoglycaemic agents, thyroid hormones, pituitary inhibitors, ovulation inducers, anti-muscarinic agents, diuretics, antidiuretics, obstetric drugs, prostaglandins, antimicrobials, anti-tuberculosis drugs, anti-malarials, antivirals, anthelmintics, cytotoxic agents, anorectic agents, agents used in hypocalcaemia, antitussives, expectorants, decongestants, bronchospasm relaxants, antihistamines, local anaesthetics, neuromuscular blockers, smoking cessation agents, insecticides, dermatological agents, nutritional agents, keratolytics, psychic-energisers, anti-acne agents, anti-itch agents and anti-cholinergic agents.

18. A method according to claim 6 wherein the membrane device comprises of an elastic, occlusive or semi-permeable layer selected from polyurethane polymers, ethylene vinyl acetate copolymers, hydrocolloid and cellulosic membranes.

19. A method according to claim 6 wherein the adhesive layer is permeable to the physiologically active agent and is selected from the group consisting of acrylics, polyethylenes, polysiloxanes, polyisobutylenes, polyacrylates, polyurethanes, plasticized ethylene vinyl acetate copolymers and tacky rubbers.
